(19) United States
(12) Patent Application Publication
(10) Pub. No.: US 2021/0169795 A1
(43) Pub. Date: Jun. 10, 2021

(54) Colloidal Suspensions of Plant Extracts in Aqueous Solutions

(71) Applicants: Joshua Steindler, Ithaca, NY (US); Robert Martinez, Ithaca, NY (US)

(72) Inventors: Joshua Steindler, Ithaca, NY (US); Robert Martinez, Ithaca, NY (US)

(21) Appl. No.: 17/114,030

(22) Filed: Dec. 7, 2020

Related U.S. Application Data
(60) Provisional application No. 62/944,882, filed on Dec. 6, 2019.

(51) Int. Cl. A61K 9/00; A23L 33/105; A61K 36/185; B01D 11/02

(52) U.S. Cl. CPC A61K 9/0095; B01D 11/02; A61K 36/185; A23L 33/105

(57) ABSTRACT

A colloidal mixture and a method for creating the same is presented comprising combining a cannabinoid or an oleoresin, having purity in the range of 0.001% to 100%, an emulsifying or stabilizing agent in sufficient amounts to suspend the cannabinoid or oleoresin within the colloidal mixture to prevent phase separation wherein the cannabinoid or oleoresin remains suspended throughout the colloidal mixture, and an acidic aqueous solution of pH less than 7.0 sufficient to dissolve the emulsifying or stabilizing agent. The combination is agitated to create a colloidal mixture wherein the cannabinoid or oleoresin remains suspended throughout the colloidal mixture.

### BACKGROUND

What is presented is a method to create a colloidal suspension of Cannabis plant extracts, including phytochemicals, alkaloids, glycosides, phenolics, flavonoids, terpenoids, terpenes, cannabinoids, oleoresins, oils, and other Cannabis plant compound(s), in an aqueous solution. The colloidal suspension can act as a carrier and delivery system for human and animal consumption or as medical treatment with increased bioavailability and be considered for use with dietary supplements or as part of digestible preparations, including food, supplement, or pharmaceutical preparations, or any other preparations utilizing Cannabis plant extract(s).

The aforementioned colloidal suspension is unique in the Cannabis industry. Hitherto, it has been impossible to stabilize cannabinoids, Cannabis oleoresins, Cannabis oils, and other Cannabis plant extracts at high concentrations in aqueous form. This method stands to revolutionize current preparations of consumable products containing Cannabis plant extracts as most or all rely on carrier oils in liquid forms or carrier starches in dry solid forms, neither of which feature aqueous solutions in an essential way.

### SUMMARY

A colloidal mixture and a method for creating the same is presented comprising combining a cannabinoid or an oleoresin, having purity in the range of 0.001% to 100%, an emulsifying or stabilizing agent in sufficient amounts to suspend the cannabinoid or oleoresin within the colloidal mixture to prevent phase separation wherein the cannabinoid or oleoresin remains suspended throughout the colloidal mixture, and an acidic aqueous solution of pH less than 7.0 sufficient to dissolve the emulsifying or stabilizing agent. The combination is agitated to create a colloidal mixture wherein the cannabinoid or oleoresin remains suspended throughout the colloidal mixture. In some embodiments, the colloidal mixture is a colloidal suspension or an emulsion. The colloidal may be developed by heating to a rolling boil, by agitation, or by sonication.

The cannabinoid or oleoresin comprises any of alkaloids, glycosides, phenolics, flavonoids, terpenoids, cannabinoids, oleoresins, plant oils, plant resins, plant extracts, plant distillates, plant isolates, and plant raw forms. The emulsifying or stabilizing agent is one of a saccharide, a protein, and a water-soluble pure solid or compound solid mixture. The acidic aqueous solution comprises one of a fruit juice, a vegetable juice, a plant juice, and purified water with added acid. The acidic aqueous solution comprises any one of citric acid, phosphoric acid, acetic acid, carbonic acid, malic acid, and tartaric acid. The cannabinoid or oleoresin is obtained from one of raw or decarboxylated plant matter, CO₂ extraction, ethanol extraction, butane extraction, butane/propane extraction, hexane extraction, or strained from plant matter soaked in a solvent mixture.

The colloidal mixture may be dried to a powder, diluted into a drink, mixed with a carrier compound into a topical compound, aerated to form a gel, boiled to a concentrate, and mixed into any food type substance, pharmaceutical, or dietary supplement.

Those skilled in the art will realize that this invention is capable of embodiments that are different from those shown and that details of the devices and methods can be changed in various manners without departing from the scope of this invention. Accordingly, the drawings and descriptions are to be regarded as including such equivalent embodiments as do not depart from the spirit and scope of this invention.

### DETAILED DESCRIPTION

Cannabis is a genus of flowering plants that includes three distinct species: Cannabis sativa, Cannabis indica, and Cannabis ruderalis. Cannabis is indigenous to Central Asia and the Indian subcontinent, and hemp, a variety of Cannabis sativa, may be one of the first plants ever cultivated, with archaeological evidence of pre-Neolithic usage in Japan. Early hemp cultivars may have been selectively bred for their fiber, used to make rope, clothes, and paper. Other civilizations took special interest in Cannabis plants for their nutritional and medicinal properties. Chinese texts as early as 100 A.D. reference the oral consumption of Cannabis in teas, which were known to induce powerful analgesic and soporific effects. Meanwhile, other strains were prized for their psychoactive properties, which were used for both religious and recreational purposes. Such usage may be traced back as early as the 8th century BCE Kingdom of Judah, where Cannabis residues have recently been recovered on two altars in Tel Arad, located in present-day Israel. Other early historical references of such usage include the Greek historian Herodotus's description of ritualistic and recreational Cannabis usage by Scythians.

“Cannabis plant(s)” refers to both wild strains and strains developed in laboratories and grow rooms. These plants contain a substantial variety of chemicals, of which 483 have so far been identified. The most abundant chemical compounds present in Cannabis are a class of organic compounds known as terpenes, which can be found in a variety of plants, most commonly in conifers. Terpenes are unsaturated hydrocarbons with the chemical formula (C₅H₈)ₙ and are the primary constituents of a plant's essential oils, serving important biological functions. In the plant kingdom, they act as powerful chemical mediators of a variety of ecological interactions. For example, terpenes are used to ward off pests and predators and also to attract mutualistic pollinators. Phytocannabinoids constitute the other major class of compounds found in Cannabis, but unlike terpenes, they are found exclusively in Cannabis. They are a class of terpenophenolic compounds made up of 21 carbons atoms. Over 113 have so far been isolated from various Cannabis cultivars. They are produced predominantly in the resin glands of the plant known as trichomes. These glandular outgrowths resemble fine hairs with a globular tip and function as a line of defense for the plant. Meanwhile, the term “cannabinoids” refers to a broader class of compounds that includes the phytocannabinoids but also structurally similar synthetic compounds, as well as endocannabinoids, a class of endogenous neurotransmitters that bind to the cannabinoid receptors found in vertebrate central and peripheral nervous systems.

Selective breeding of Cannabis has led to a variety of cultivars that have specific pharmacological profiles, which can be attributed to their unique chemical make-up, in particular their phytocannabinoid composition. For example, some strains contain a minimal amount of THC, more formally known as Δ9-tetrahydrocannabinol, the psychoactive chemical that is responsible for the intoxication for which Cannabis is best known. Meanwhile, other agricultural approaches have gone in the opposite direction, producing THC-rich plants that ensure a highly psychoactive experience.

Aside from THC, the most abundant cannabinoid in Cannabis is cannabidiol, or CBD. THC is psychoactive, while CBD is known for its anti-inflammatory, anti-convulsant, and pain-relieving properties. Cannabis plants are categorized depending on the amount of THC they produce, as well as the ratio of THC to CBD they contain. “Drug plants” produce high levels of THC and low levels of CBD levels while “non-drug plants” produce a THC:CBD ratio that is significantly lower.

Apart from THC and CBD, other known and well-studied cannabinoids include Cannabinol (CBN), Cannabichromene (CBC), Cannabigerol (CBG), Cannabinodiol (CBND), Cannabicyclol (CBL), Cannabivarin (CBV), Tetrahydrocannabivarin (THCV), Tetrahydrocannabiphorol (THCP), Cannabidivarin (CBDV), Cannabichromevarin (CBCV), Cannabigerovarin (CBGV), and Cannabigerol Monomethyl Ether (CBGM). 2-carboxylic acid variations of each of these cannabinoids (in their 2-COOH form) are biosynthetic precursors that are converted to their respective active cannabinoid compounds through decarboxylation, which can be catalyzed by alkaline conditions, light, or heat. The nomenclature for the acidic precursors is relatively simple: name the Cannabis compound the precursor forms and add an “A” after it. For example, the acidic precursor of THC is THCA, the acidic precursor for CBD would be CBDA, and the precursor for CBC would be CBCA, and so on. In most cases, cannabinoids only exhibit their active properties once they have been decarboxylated; the acidic precursor for THC for instance, THCA, is not psychoactive.

THC has a similar chemical structure to the fatty acid neurotransmitter anandamide, a naturally occurring endocannabinoid. For this reason, both anandamide and THC bind to CB1, a G protein-coupled cannabinoid receptor present primarily in the central nervous systems of vertebrates, with high affinity. While anandamide does produce a mildly euphoric effect in humans, THC elicits the much more pronounced psychoactive effect for which Cannabis is known. Furthermore, through its interaction with the endocannabinoid receptor system of the CNS, THC can elicit a plethora of neuroprotective and analgesic responses, such as the promotion of neurogenesis and the reduction of inflammation in the brain. THC binds with almost equal affinity to cannabinoid receptors CB1 and CB2, the two known subtypes of cannabinoid G-couple protein receptors present in the human brain and body. CB1 receptors are expressed predominantly in the central and peripheral nervous system, while CB2 receptors are expressed more abundantly in the immune and digestive systems.

In contrast to THC, CBD is not psychoactive. In fact, recent studies suggest CBD mitigates the adverse effects of THC, especially in individuals experiencing schizophrenia-like symptoms. Furthermore, unlike THC, CBD does not bind with a high affinity to either CB1 or CB2 receptors, though it does show a higher affinity to CB2 than it does to CB1. Instead, CBD acts as an indirect antagonist of cannabinoid agonists, and it also acts as an effective agonist for 5-HT1A receptors, which are known to mediate processes linked to depression, anxiety, and protection of neural development. Furthermore, CBD has been shown to reduce inflammation, nausea, anxiety, and seizures and seems to also help in reducing the memory loss associated with THC consumption in mammals.

Cannabinol (CBN) is a metabolite formed from the degradation of THC. There is very little CBN present in a fresh Cannabis plant. As THC degrades in storage over time while exposed to air and light, CBN content increases. Like CBD, CBN exhibits a higher affinity to CB2 receptors than to CB1 receptors and, unlike THC, is only mildly psychoactive. Cannabigerol (CBG) mildly affects the general effects of the full Cannabis plant; it acts as a 5-HT1A and CB1 receptor antagonist, binds to CB2 receptors, and is an agonist for the α2-adrenergic receptor. A now-denominated CB3 receptor has also recently been discovered, but its exact mechanism is still unknown. Tetrahydrocannabivarin (THCV) is only known to be an antagonist of THC at CB1 receptors; like CBD, it seems to lessen THC's psychoactive effects. Cannabichromene (CBC) is known to be anti-inflammatory, analgesic, and is non-psychoactive.

What is described herein is a novel method to create a colloidal suspension of Cannabis plant extracts of any concentration and purity in an acidic aqueous solution. The method could be applied, in particular, to Cannabis plant cannabinoids, oleoresins, and oils for improved bioavailability in consumed preparations. The emulsifying or stabilizing agent in the colloidal mixture acts to slow phase separation of the heterogenous mixture, which gives the colloidal suspension added stability and a relatively long shelf life. The fact that such a mixture may be created by using fruit or vegetable juices as the chosen acidic aqueous solvent and primary carrier yields a new and innovative alternative to known Cannabis plant-containing products currently on the market and allows for a myriad of equally novel derivatives.

Due to the novel method to prepare said colloidal suspension, the bioavailability of the relevant Cannabis plant extracts is significantly improved when compared to other preparations containing equivalent dosages currently available on the commercial market. This is due to the microscopic size of the resulting suspended globules of the Cannabis plant extracts which accelerates their transport from the alimentary system into the bloodstream bypassing digestion and, therefore, into the endocannabinoid and central nervous systems or other bodily systems, where they reach the appropriate receptors.

This method produces a colloidal suspension of Cannabis plant extracts which may be reduced, diluted, or otherwise incorporated or combined into any and all forms of substances including tinctures, beverages, pills, foods, supplements, medications, concentrates, gels, lozenges, powders, or any other product and could be classified as a Botanical Drug Substance (BDS) for medical and pharmaceutical uses, in particular. Given the emerging medical, pharmaceutical, and recreational Cannabis markets, including the current medical applications of Cannabidiol (CBD), colloidal suspensions of Cannabis plant extracts with high bioavailability would be a suitable and possibly superior carrier and delivery system on said markets. In fact, all known BDSs involving any Cannabis plant extracts are compatible with this method.

Cannabinoids are Cannabis plant extracts and, therefore, are compatible with this method, in particular, and include but are not limited to Tetrahydrocannabinol (THC), CBD, Cannabinol (CBN), Cannabigerol (CBG), Cannabidiolic acid (CBDa), Tetrahydrocannabinolic acid (THCa), Tetrahydrocannabivarin (THCV), Cannabichromene (CBC), etc., thereby giving rise to new therapeutic pathways for the future of medical applications of said compounds. A growing body of research suggests that certain cannabinoids have specific therapeutic actions against various diseases and derangements, although the primary mechanisms remain an area of active medical research. Similarly, certain terpenoids and terpenes for example, may increase the physiological activity of known BDSs, especially those found in mint, mango, lavender, peppermint, and the Cannabis plant. Compounds such as L-theanine, piperine, valerian alkaloids, kava, sesquiterpenes, oxytocin, theobromine, passionflower, melatonin, GABA, catechins, valeric acid, and other naturally occurring plant compounds may also change the functional effects of BDSs.

The resulting colloidal suspension may be used to create beverages containing Cannabis plant extracts. It also could be used to form topicals, gels, creams, powders, perfumes, foods, drug delivery systems, etc., which are essential vehicles in medicine, pharmaceuticals, food and beverages, health and wellness, exercise and fitness, and personal care and beauty. In particular, the benefits from the disclosure herein are as abundant as the well-documented and varied advantages that have been recorded from Cannabis use and consumption: medical conditions as varied as chronic pain disorders, anxiety disorders, sleep disorders, Post Traumatic Stress Disorder (PTSD), drug dependence, Crohn's disease and Irritable Bowel Syndrome (IBS), paralysis, neuropathy, autism spectrum disorder, cancer, epilepsy and seizure disorders, high blood pressure, and glaucoma among other maladies have been shown to respond positively to varying degrees of Cannabis exposure. In many instances, patients who suffer from any of these conditions or other conditions are physically unable to ingest Cannabis products through more conventional means such as smoking, and both young or elderly patients can benefit from the ease of consumption through liquid and solid food formulations. The circumnavigation of the digestive system by the colloidal mixtures disclosed herein allows for rapid onset of beneficial effects of the Cannabis plant extracts not unlike smoking but without the complications of any pulmonary harm.

What is presented herein is a solution, reduction, and a method of making a liquid solution in the form of a colloidal mixture and/or a solid, syrup liquid, or gel like reduction of the same comprising a water-based acidic solution (such as fruit and vegetable juice), Cannabis oleoresin(s) or oils, and an emulsifying or stabilizing agent, which may or may not be added to the preparation depending on the source acidic solution which may already include an emulsifying or stabilizing agent. Cannabis oleoresins, for example, may be specific forms of Cannabis plant extracts as well as oils, resins, extracts, distillates, isolates, raw forms (flower or plant material), and acidic variants. The acidic aqueous solution may be any juice from a plant, vegetable, or fruit that contains acid. The acidic aqueous solution can be any aqueous liquid preparation with additional solid or liquid acid. The emulsifying or stabilizing agent may be sucrose, dextrose, fructose, maltose, or any other sugar, including sugar alcohols, agave, honey, and other natural sugars and sweeteners. A solution of water or other liquid or substance and the aforementioned ingredients may be combined with other compounds for a variety of purposes or actions.

Hitherto, a delivery system for Cannabis plant extracts and oleoresins using aqueous solutions has not been developed; the compositions that are currently available are overly complicated or contain compounds that do not produce a colloidal suspension. Some current compositions require the use of emulsifiers or starches, which are not desirable in certain circumstances and may not mix easily or dissolve completely in aqueous solutions. Prior art compositions use starches or cyclodextrins in dry powder form for hot beverages and the like or emulsifiers like xanthan gum, guar gum, cyclodextrin, lecithin, carrageen, monoglycerides, natural emulsifiers and organic emulsifiers.

What is presented herein is a simple method of dissolving Cannabis plant extracts, including in particular, cannabinoids and other Cannabis oleoresins and oils, in aqueous solutions to form a colloidal mixture using simple ingredients. What is claimed is a method to achieve such colloidal mixtures that is not intuitive given known hydrophobic properties of Cannabis plant extracts.

The current disclosure addresses the need for an accessible, simple, and shelf-stable delivery system for preparation and consumption of Cannabis plant extracts. Innovating upon current approaches and optimizing the presently available knowledge, the produced colloidal mixtures allow for selective and safe stimulation of the endocannabinoid system without compromising potency. The composition produced herein, and its multiple possible presentations disclosed herein, provide products that can be tailored for a variety of purposes. Such products could further destigmatize Cannabis consumption in populations that may not look favorably on other forms of ingestion such as through smoking. Oral ingestion through beverages is significantly more discreet and socially acceptable.

The method disclosed herein was developed while experimenting with various carrier and delivery systems to improve the bioavailability of Cannabis plant extracts. The proposed colloidal mixture provides a novel substance with specific physiological properties that are mediated through separate receptor pathways and provide numerous advantages and benefits to the consumer.

The method for creating a colloidal mixture comprising the steps of combining: 1) a cannabinoid or an oleoresin, having purity in the range of 0.001% to 100%; 2) an emulsifying or stabilizing agent in sufficient amounts to suspend the cannabinoid or oleoresin within the colloidal mixture to prevent phase separation wherein the cannabinoid or oleoresin remains suspended throughout the final colloidal mixture; and 3) an acidic aqueous solution of pH less than 7.0 sufficient to dissolve the emulsifying or stabilizing agent. The combination is agitated to create a colloidal mixture wherein the cannabinoid or oleoresin remains suspended throughout the colloidal mixture.

The cannabinoid or oleoresin may comprise alkaloids, glycosides, phenolics, flavonoids, terpenoids, cannabinoids, oleoresins, plant oils, plant resins, plant extracts, plant distillates, plant isolates, and plant raw forms. These compounds may themselves act as emulsifying or stabilizing agents or not, act as psychoactive compounds affecting and interacting with the endocannabinoid system, or on other known or unknown biological systems in the human body through known or unknown physiological mechanisms. Naturally occurring in unrefined Cannabis plant extracts, terpenoids and terpenes are believed to enhance bioavailability and effect.

The emulsifying or stabilizing agent can be a saccharide, protein, or any other water-soluble pure solid or compound mixture. All fruit or vegetable juices contain saccharides in the form of simple sugars and many are also acidic. Citric acid, phosphoric acid, acetic acid, carbonic acid, malic acid, and tartaric acid or any other suitable acid can be used instead of a fruit or vegetable juice to acidify a starting aqueous solution such as simple distilled water. It should be noted that we have observed that to achieve a colloidal suspension of Cannabis plant extracts of high concentration, any amount of emulsifying or stabilizing agent should be added to the mixture to start the emulsification process.

Heating and agitation are the primary ways that a colloidal suspension can be created, but colloidal suspension can also be achieved by sonication or other vibration of the ingredients. The cannabinoid or oleoresin can be obtained from any form and concentration of Cannabis plant extracts, including raw or decarboxylated plant matter. Such Cannabis plant extracts may be performed by any extraction methods including by using CO₂ extraction, ethanol or other organic solvent, butane extraction, butane-propane extraction, strained from plant matter soaked in a solvent mixture or by another extraction method. A microwave, which is readily available in many kitchens, is a suitable device for heating and agitating the mixture to create the colloidal suspension.

The aforementioned colloidal mixture is versatile. Once formed, it can be added to any other liquid and made into a beverage itself, made into a topical compound, made into a gel, concentrated by further heating for later reconstitution or not, dried into a powder to be reconstituted or not, or mixed as is or in any of these forms into any food or water-based preparation, including pharmaceutical or dietary supplements. The size of the suspended globule of cannabinoid or oleoresin in the colloidal suspension can be controlled by varying the ratios of ingredients and the level and duration of heat and agitation.

An example of a recipe to create a colloidal mixture disclosed herein is one combining Cannabis plant extract(s) with a suitable fruit or vegetable juice or acidic mixture (such as tomato, grapes, orange, lime, lemon, mango, pineapple, etc.), water, and sugar (such as table sugar, sucrose, dextrose, fructose, maltose). The mixture is microwaved or otherwise heated and/or agitated until the solution forms a colloidal suspension. Sugar could be omitted or limited if the starting solution is a sweet fruit or vegetable juice. Other formulations that are similar do work and should be considered depending on available resources.

A specific example of creating the colloidal mixture disclosed herein with concrete quantities is as follows: Mix 4 grams of lime juice concentrate with 2 grams of table sugar. Add 300 mg of distillate of Cannabis plant extract of 80% purity. Microwave the mixture for 1 minute on ‘High’ setting. The mixture will come to a rolling boil and form a colloidal suspension. Due to water evaporation, there will be approximately 4 grams of mixture remaining, of which 300 mg will be distillate of Cannabis plant extract. The colloidal suspension can then be diluted in sparkling water to create a beverage or dried in a dehydrator at 170 degrees Fahrenheit for 24 to 48 hours to a powder form for transfer into capsules for dietary supplementation.

As mentioned previously, Cannabis plants naturally produce a class of chemical compounds known as terpenes. Every Cannabis strain is known for its particular terpene profile, and over 100 terpenes have been identified in Cannabis. Terpenes synergize with cannabinoids to create their effects. Terpenes can be added to the disclosed colloidal mixtures formulated herein to increase bioavailability or variety or to simply offer a different odor/flavor profile. Some of the best known terpenes are listed below, in alphabetical order:

1. Borneol is easily synthesized into menthol; it has a woody, pine-like, minty odor and is considered a sedative. It is recommended for ailing stress, fatigue, and helps with recovery from sickness.

2. Caryophyllene is known to block potassium and calcium channels in high amounts, which are essential for heart muscle control. It also has a weak affinity to CB2 receptors and has a peppery, clove-like, sweet and spicy smell.

3. Cineole (Eucalyptol) gets its name from its similarity in odor to eucalyptus, which is refreshing and minty with spicy undertones. It aids with pain relief and circulation.

4. Delta-3-Carene is found in cypress oil, which is popular in aromatherapy. It has a pine like, woody and pungent smell, and it aids in drying excess fluids like menstrual blood, sweat, tears and mucus.

5. Limonene, as its name suggests, is citrus-like in odor, together with hints of juniper, peppermint and rosemary. It repulses predators and is naturally found in many flowers and fruit rinds. In conjunction with other terpenes, limonene can promote other terpenes' absorption and also exhibits anticarcinogenic, anti-fungal, antidepressant, and anti-bacterial properties.

6. Linalool is anxiolytic and sedative with a floral and citrus odor.

7. Myrcene is the most prevalent terpene in Cannabis and is an important component for other compounds like geraniol, citronella, and menthol. It is antimicrobial, analgesic, antioxidant, antidepressant, muscle-relaxant, anti-inflammatory, and antiseptic. It smells clove-like, with earthy, citrus and fruity undertones, including hints of mango and mint. It is also known to affect cell permeability: more THC can reach the cell when myrcene is present.

8. Pinene smells pine-like, as its name suggests, with hints of rosemary in its Alpha variety and dill, parsley, and basil in its Beta presentation. It increases energy and mental focus and is a bronchodilator, topical antiseptic, and expectorant. Pinene also aids memory retention by destroying acetylcholine and can counteract THC activity in the brain.

9. Pulegone inhibits acetylcholinesterase, which is used in the brain for memory storage, and has a minty and rosemary-like odor.

10. Terpenes and related compounds including anthocyanins can easily be fractionated using steam distillation. These terpenes can be reserved as the plant material is processed and reincorporated into the mixture at will, giving the manufacturer control over profiles.

Methods for calculating cannabinoid content in assays are well known in the industry, and this disclosure, to a varying degree, provides extracts that have predefined cannabinoid ratios. The extracts cannabinoids and oleoresins used herein can be derived from acceptable carriers. As a result, the present disclosure not only provides a new BDS but also provides a method to create a substance that is highly bioavailable as a pharmaceutically active cannabinoid compound.

It is viable to prepare separate extracts from single Cannabis varieties for different cannabinoid content and then blend or mix them together to have a final product with specific pharmacological properties; as an alternative, different plants can be blended or mixed and then extracted together. In a similar fashion, pharmaceutically acceptable carriers, diluents or excipients can be used depending on the targeted mode of administration of the final product, which can be liquids or liquids meant to be pumped as aerosol sprays, pills, tablets, gels, capsules, powders, suppositories, vaporizers and nebulizers, food products, etc.

The method disclosed herein can be utilized to obtain a cannabinoid-rich substance from Cannabis plant material. The method includes extraction of cannabinoids from provided Cannabis plant material, winterizing and purging said extract and optionally decarboxylating said extract prior to winterization or extraction. Cannabinoid acid decarboxylation depends on time and temperature: higher temperatures result in a shorter decarboxylation time. There are important considerations when selecting the right conditions for acid decarboxylation. For example, if the decarboxylation temperature is too high, thermal degradation can destroy the Cannabis compounds desired, leading to, for example, the degradation of THC into CBN. An ideal process for decarboxylation is to heat dried plant material for at least 15 minutes at a temperature of 220 degrees Fahrenheit and then to heat for at least 45 minutes at 285 degrees Fahrenheit. In some instances, the 285-degree heating step is increased to 45 to 75 minutes and is then followed by an additional heating cycle of 50-90 minutes at 294 degrees Fahrenheit.

Particularly suited acids for the colloidal mixtures presented herein include but are not limited to: Lactic Acid, Acetic Acid, Formic Acid, Citric Acid, Oxalic Acid, Uric Acid, Malic Acid, Phosphoric Acid, Tartar Acid, and any other acid known. Some of these acids may be found naturally occurring in fruit and vegetable juices, which also contain saccharides and simple sugars.

Well-suited juices for the acidic aqueous solution include but are not limited to: Aloe Vera Juice, Apple Juice, Beet Juice, Bhang, Cantaloupe Juice, Carrot Juice, Celery Juice, Chanh Mu\hat{ó}i , Cherry Juice, Clam Juice, Cranberry Juice, Coconut Water, Cucumber Juice, Dandelion-Green Juice, Grape Juice, Grapefruit Juice, Guava Juice, Honeydew Juice, Izze, Jungle Juice (Alcoholic Beverage), Kaffir Lime Juice, Kiwifruit Juice, Lemonade, Lemon Juice (reconstituted or fresh), Limeade, Limonana, Lychee Juice, Mango Juice, Melon Juice, Mora (drink), Grapes, Lassi, Orange Juice, 'Otai, Papaya Juice, Parsley Juice, Petimezi (Grapefruit), Pineapple Juice, Pog (passionfruit, orange, guava), Pomegranate Juice, Prune Juice, Raspberry Vinegar, Concentrated or Fresh Lime Juice, Şalgam (carrots), Spinach Juice, Strawberry Juice, Sugarcane Juice, Tejuino Fermented Corn, Tomato Juice, and Turnip Juice or juice from any other fruit or vegetable.

Well-suited sugars for the emulsifying or stabilizing agent include but are not limited to: Agave Nectar, Arabinose, Barbados Sugar, Barley Malt Syrup, Barley Malt, Barley Sugar, Beet Sugar, Birch Syrup, Brown Sugar, Buttered Syrup, Cane Sugar, Carob Syrup, Caster Sugar, Coconut Sugar, Confectioner's Sugar, Corn Sugar, Corn Syrup, Date Sugar, Dehydrated or Liquid Cane Juice, Demerara Sugar, Dextrin, Dextrose, Disaccharides, Evaporated Cane Juice, Free Sugar, Fructose, Fucose, Galactose, Glucose, Glucose Solids, Golden Syrup, Golden Sugar, Grape Sugar, High Fructose Corn Syrup, High Maltose Corn Syrup, Honey, Inositol, Inverted Sugar Syrup, Jaggery, Lactose, Malt Extract or Malt Syrup, Maltose, Maltodextrin, Maltol, Mannose, Maple Sugar, Maple Syrup, Molasses, Monosaccharide, Muscovado, Non-centrifugal Cane Sugar, Palm Sugar, Penuche, Powdered Sugar, Raw Sugar, Refiner's Sugar, Refiner's Syrup, Ribose, Rice Syrup, Rhamnose, Saccharose, Sorghum Syrup, Sucrose, Sugar beet juice, Sugarcane, Sweet Sorghum, Syrup, Toffee, Treacle, Trehalose, Yellow Sugar, Xylose, and any other saccharide and sugar.

Particularly suited additives include; Mint, Mango, Lavender, Peppermint, L-theanine, Piperine, Valerian Alkaloids, Kava, Sesquiterpenes, Oxytocin, Theobromine, Passion Flower, Melatonin, GABA, Vitamins, Minerals, Proteins, Oils, Nutritional products, Chocolate, Vanilla, Catechins, Valeric Acid, Melatonin, Terpenes, Juices, Seltzers, Carbonated Beverages, Syrups, Essential Oils, Calcium, Echinacea, Ginseng, Glucosamine and/or, Chondroitin Sulphate, Garlic, Vitamin D, Sulforaphane, Curcumin, St. John's Wort, Saw Palmetto, Ginkgo, Green Tea, Coenzyme Q10, Folic Acid, Flavors (artificial and natural) and any other plant or artificial additives.

### Sample Preparations and Recipes

### EXAMPLE 1

### Cannabis Plant Preparation and Storage

Flower buds alone or flower buds and leaves from fresh Cannabis plants are harvested from either cuttings that are taken from mother plants, a seed source from which the Cannabis plant is grown, or tissue culture. In all three instances, the result will be a Cannabis plant that has specific starting ratios that are maintained for as long as the plant continues to flower. The plant material is first harvested and immediately frozen, preferably flash-frozen between −50- and 10-degrees Fahrenheit for ten minutes and stored in vacuum-sealed bags for at least 36 hours before the plant material is extracted. In this way, the starting Cannabis plant material will be extracted at roughly 80-90% concentration.

### EXAMPLE 2

### Inactive Cannabinoid Extraction

Flash-frozen or dried Cannabis flowers (See Example 1) are spread on curing screens (150-200 μm), slightly broken apart to achieve a uniform spread of small-sized pieces that are less than half an inch in length to be dried out. After the plant material has dried, currently inactive from not having undergone decarboxylation, it is transferred into a closed-loop hydrocarbon extractor. This can be either a closed-loop butane system or a supercritical CO₂ extractor. Alternatively, a simple salt-water or water extraction technique where the material is mixed with ice and water and filtered through silkscreen could be used. Pressed rosin methods also work as well as other known extraction method.

The material extracted from this process can be winterized to filter out any waxes. Winterization can be achieved by introducing ethanol to the extract and storing the mixture between −80 to −20 degrees Fahrenheit for at least two days. After this time, the waxes can be filtered by passing the material through a 1-20 μm membrane. The extract can be further clarified using all known methods of the art. Alternatively, it can be dewaxed/winterized inline during the extraction process through a jacketed column of plant material, usually surrounded by a mixture of alcohol and dry ice with a micron filter collecting the waxes. Although this is less efficient, it preserves more volatile compounds. Subsequently, the extract can be purged under a vacuum oven with a solvent-rated pump (minimum ½ hp, 3,425 rip oil-less compression). The vacuum drying oven with a solvent-rated recovery pump can remove excess solvent from the extract when used over two days. The resulting product is removed from the oven and stored away from light and under refrigeration until needed for preparations.

### EXAMPLE 3

### Active Cannabinoid Extraction

Air-dried Cannabis flowers (see Example 2) are placed in a sterile containment oven for 15 minutes at 220 degrees Fahrenheit and then for 45 minutes at 280 degrees Fahrenheit. This process will decarboxylate the cannabinoids present in the plant material, and the material is considered at a nonacid stage or active after this process. The active plant material can then be transferred into a closed-loop extractor and held at the native pressure of the butane, propane or other hydrocarbons in the system or to system specifications.

The material extracted from this process can be winterized to filter out any waxes, which can be achieved by introducing ethanol to the extract and storing it between −20 to −80 degrees Fahrenheit for at least two days. After it has been stored, the waxes can be filtered out by passing the material through a 1-20 μm membrane. The extract can be further clarified using all known methods of the art. As the final step, the resulting product is purged under the vacuum oven for another two days. Alternatively, it can be dewaxed/winterized inline during the extraction process through a jacketed column of plant material, usually surrounded by a mixture of alcohol and dry ice with a micron filter collecting the waxes though this is less efficient it preserves more volatile compounds.

All of the preparations below involve an ingredient of a colloidal suspension of Cannabis plant extract(s) as outlined above:

### EXAMPLE 4

### Beverages

1. Add a measure of the colloidal mixture prepared as disclosed to a beverage of choice as needed for desired dosage.

### EXAMPLE 5

### Concentrate Additive For Beverages, e.g. Flavor Enhancer

1. Concentrate the colloidal mixture prepared as disclosed to desired dosage by varying ratios during the production procedure.

2. Take the colloidal mixture, add water until desired consistency is achieved.

3. Any suitable preservative may be added or not, depending on the solution contents.

4. Any suitable flavor may be added or not.

One could take the colloidal mixture and add it to pure water as a carrier and delivery system for Cannabis plant extracts and flavoring, making a useful product that is easily customized by the consumer with, for example, CBD, which is known to be safe at high dosages.

### EXAMPLE 6

### Liquid Capsule

1. Concentrate the colloidal mixture prepared as disclosed to desired dosage by varying ratios during the production procedure.

2. Any suitable preservative may be added or not depending on the solution contents.

3. Use any commercial or small scale gelatin or other material liquid capsule type machine to encapsulate portions of the colloidal mixture and form pills.

Giving a very stable and controlled dosage, this method would make a faster acting pill available for any solution recipe that could be taken in a pill form, as most cannabinoids, Cannabis oleoresins, and Cannabis oils are usually, if not always, put into an oil-based carrier. A water-based carrier is faster absorbing.

### EXAMPLE 7

### Powdered Pill

1. Dehydrate the colloidal mixture prepared as disclosed using any dehydration method after controlling dosage and consistency during production process.

2. Pulverize, grind or otherwise turn the dehydrated solution into a powder with any means that satisfy the need.

3. Add or do not add said powder to a commercial excipient or combination of excipients and binders

4. Encapsulate powder in pill form in any manner.

Most pharmaceutical capsules use dry starches as carriers that are poorly absorbed along with substances like lactose, cellulose, magnesium stearate etc., as fillers. This is a much more desirable formulation in terms of using the solution dehydrated versus any other known carrier in this situation.

### EXAMPLE 8

### Pressed Tablets

1. Dehydrate the colloidal mixture prepared as disclosed using any dehydration method.

2. Pulverize, grind or otherwise turn the dehydrated solution into a powder with any means that satisfy the need.

3. Add said powder to a commercial excipient.

4. Press the powder into a pill of desired shape using any suitable manner.

### EXAMPLE 9

### Topical Lotion, Cream, or Gel

1. Concentrate the colloidal mixture prepared as disclosed to desired dosage by varying ratios during the production procedure.

2. Add said solution to any suitable cream, lotion, gel, or transdermal recipe (e.g. Simple balm recipe: 1½ cups of coconut oil, ⅓ cup olive oil (optional), ⅓ cup beeswax. Combine with heat, add the colloidal mixture prepared as disclosed in either liquid or dehydrated form, optionally add essential oils for fragrance or effect)

This process would allow the colloidal mixture to be added to any cream, gel, lotion or transdermal (that is safe) for commercial or home-based kit use.

### EXAMPLE 10

### Cosmetic Products

1. Concentrate the colloidal mixture prepared as disclosed to desired dosage by varying ratios during the production procedure.

2. Add the colloidal mixture at desired concentration to any cosmetic product.

Recently, CBD cosmetics have become a trend. While there is little evidence for beneficial effects beyond possible antioxidative effects, this process could combine an absorbable formula of BDS with any suitable cosmetic product.

### EXAMPLE 11

### Dog or Cat Food or Similar

1. Concentrate the colloidal mixture prepared as disclosed to desired dosage by varying ratios during the production procedure.

2. Add the colloidal mixture during manufacture of commercial animal food either in liquid or dehydrated form.

CBD has been used increasingly as an ingredient in dietary products meant for animal consumption. Since the solution increases CBD's bioavailability, its use in animal food may enhance CBD delivery to the consumer.

### EXAMPLE 12

### A Powder For Later Reconstitution

1. Dehydrate the colloidal mixture prepared as disclosed with any method that works and crush to a fine powder with any method that works.

2. Add water to reconstitute to a stable drink with or without other ingredients.

This version is perfect for drinks or nutritional beverages where reconstitution of the beverage from a dried powder is desirable.

### EXAMPLE 13

### A Dehydrated Nutritional Drink

1. Dehydrate the colloidal mixture prepared as disclosed with any method that works and crush to a fine powder with any method that works.

2. Add nutrients including proteins and fats (possibly from medium-chain triglyceride (MCT) oil in powder form)

3. Additional plant phytonutrients such as polyphenol containing ingredients, anthocyanins, antioxidants, extracts from common herbs, roots and plants etc.

4. Vitamins and Minerals may be added or not or any other nutritionally or functional substance.

### EXAMPLE 14

### Coffee, Fresh

1. Concentrate the colloidal mixture prepared as disclosed to desired dosage by varying ratios during the production procedure.

2. Spray on coffee beans by any useful method and dry by any means useful.

### EXAMPLE 15

### Coffee, Instant

1. Concentrate the colloidal mixture prepared as disclosed to desired dosage by varying ratios during the production procedure.

2. Dehydrate the colloidal with any method that works and crush to a fine powder with any method that works.

3. Mix into any instant form of coffee that is useful (including pod type coffees).

### EXAMPLE 16

### Candy, Hard

1. Dissolve sugar in water/liquid per recipe instructions and heat (E.g. Cook sugar to “hard crack” temperature, 295-309 degrees F., add flavoring and color in a well-ventilated area).

2. Add the colloidal mixture prepared as disclosed into the sugar/water solution prior to, or after, reaching desired temperature.

3. Heat until desired consistency, pour into molds, let cool.

### EXAMPLE 17

### Candy, Soft

1. Dissolve sugar in water/liquid per recipe instructions (E.g. Cook sugar to “soft crack” temperature, 270-289 degrees F., add flavoring and color in a well-ventilated area, gelatin and other additives may be used to achieve desired consistency)

2. Add the colloidal mixture prepared as disclosed into solution prior to, or after, reaching desired temperature.

3. Heat until desired consistency, pour into molds, let cool.

### EXAMPLE 18

### Lollipops

1. Dissolve sugar in water/liquid per recipe instructions (E.g. Cook sugar to “hard crack” temperature, 295-309 degrees F., add flavoring and color in a well-ventilated area).

2. Add the colloidal mixture prepared as disclosed into solution prior to, or after, reaching desired temperature.

3. Heat until desired consistency, pour into molds with sticks, let cool.

### EXAMPLE 19

### Gummies

1. Combine Granulated Sugar, Water or flavored liquid like juice, Glucose, Sorbitol, Gelatin, Citric Acid, Candy Flavoring as recipe demands.

2. Add the colloidal mixture prepared as disclosed into solution prior to, or after, reaching desired temperature as the mixture is cooked.

3. Heat until desired consistency, pour into molds, let cool.

### EXAMPLE 20

### Breath Mints

1. Fold the colloidal mixture prepared as disclosed into gum paste prior to extending it and molding the mints; follow recipe instructions.

### EXAMPLE 21

### Sauces

1. Add I the colloidal mixture prepared as disclosed to water until desired dosage is achieved.

2. Incorporate into sauce recipe by any means useful, usually just by adding the colloidal mixture.

### EXAMPLE 22

### Baked Goods

1. Add the colloidal mixture prepared as disclosed to water until desired dosage is achieved.

2. Add to liquid mixture phase of baked goods or incorporate at any stage prior to complete mixture and baking.

3. Alternatively, the colloidal mixture can be sprayed into/onto finished product after baking.

### EXAMPLE 23

### Mouthwash

1. Add the colloidal mixture prepared as disclosed to water until desired dosage is achieved.

2. Add colloidal mixture/water mixture into any desired mouthwash recipe.

### Example 24

### Chocolate

1. Dehydrate the colloidal mixture prepared as disclosed using any dehydration method.

2. Pulverize, grind or otherwise turn dehydrated solution into a powder with any means that satisfy the needs.

3. Add said powder to any chocolate mixture prior to tempering.

4. Mix thoroughly.

5. Temper chocolate to desired consistency in a candy mold or other suitable form.

### EXAMPLE 25

### Bath Bombs

1. In a large bowl, mix baking soda, citric acid, cornstarch, and salt until well blended with no lumps.

2. Mix in olive oil and essential oil until well blended. At this point add the colloidal mixture prepared as disclosed.

3. Spray the mixture with witch hazel until it is moist enough to form into shapes that hold. Be careful not to add too much moisture or the mixture will begin to fizz. Moisture is the essence of wetness, and wetness is the essence of beauty.

4. Press mixture into molds; let sit for 5-10 minutes, then carefully remove from molds onto parchment paper and let dry for 24 hours.

### EXAMPLE 26

### Granola

1. Combine 3 cups rolled oats, 1 cup slivered almonds, 1 cup cashews, ¾ cup shredded sweet coconut, ¼ cup plus 2 tablespoons dark brown sugar, ¼ cup plus 2 tablespoons maple syrup, ¼ cup vegetable oil, and ¾ teaspoon salt.

2. Add the colloidal mixture prepared as disclosed with the desired dosage.

3. Bake in an oven at 250 degrees Fahrenheit until mixture is dry.

4. Add raisins or other dried fruit.

### EXAMPLE 27

### Snack Foods

1. Lay out any snack food on parchment paper, etc.

2. Add the colloidal mixture prepared as disclosed with the desired dosage.

3. Dry by any means necessary.

4. Test using standard lab testing (e.g. HPLC) to ensure correct dosage.

### EXAMPLE 28

### Tincture

1. The colloidal mixture prepared as disclosed can simply be consumed as a tincture, in dropper bottles or administered in any other controlled way

2. A preservative may be added if needed.

The simplest way to use the colloidal mixture is also possibly the area where there is currently a similar product that is less effective and quite ubiquitous. CBD tinctures are sold in almost every store and rely mostly on MCT oil. The colloidal mixture prepared as disclosed would serve as a healthier and more bioavailable replacement for these products.

### EXAMPLE 29

### A Food/Drink Additive

1. Dehydrate the colloidal mixture prepared as disclosed using any dehydration method.

2. Pulverize, grind or otherwise turn dehydrated solution into a powder with any means that satisfy the needs.

3. Add to any food or drink as needed.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

1. A colloidal mixture, comprising:
a cannabinoid or an oleoresin, having purity in the range of 0.001% to 100%;
an emulsifying or stabilizing agent in sufficient amounts to suspend said cannabinoid or said oleoresin within said colloidal mixture to prevent phase separation wherein said cannabinoid or said oleoresin remains suspended throughout said colloidal mixture; and
an acidic aqueous solution of pH less than 7.0 sufficient to dissolve said emulsifying or stabilizing agent.

2. The colloidal mixture of claim 1 further comprising said cannabinoid or said oleoresin comprises any of alkaloids, glycosides, phenolics, flavonoids, terpenoids, cannabinoids, oleoresins, plant oils, plant resins, plant extracts, plant distillates, plant isolates, and plant raw forms.

3. The colloidal mixture of claim 1 further comprising said colloidal mixture is a colloidal suspension or an emulsion.

4. The colloidal mixture of claim 1 further comprising said emulsifying or stabilizing agent is one of a saccharide, a protein, and a water-soluble pure solid or compound solid mixture.

5. The colloidal mixture of claim 1 further comprising said acidic aqueous solution is one of a fruit juice, a vegetable juice, a plant juice, and purified water with added acid.

6. The colloidal mixture of claim 1 further comprising said acidic aqueous solution comprises any one of citric acid, phosphoric acid, acetic acid, carbonic acid, malic acid, and tartaric acid.

7. The colloidal mixture of claim 1 further comprising said colloidal mixture is developed by heating to a rolling boil.

8. The colloidal mixture of claim 1 further comprising said colloidal mixture is developed by agitation or sonication.

9. The colloidal mixture of claim 1 further comprising the cannabinoid or oleoresin is obtained from one of raw or decarboxylated plant matter, CO₂ extraction, ethanol extraction, butane extraction, butane/propane extraction, hexane extraction, or strained from plant matter soaked in a solvent mixture.

10. The colloidal mixture of claim 1 further comprising, said colloidal mixture is one of dried to a powder, diluted into a drink, mixed with a carrier compound into a topical compound, aerated to form a gel, boiled to a concentrate, and mixed into any food type substance, pharmaceutical, or dietary supplement.

11. A method for creating a colloidal mixture comprising the steps of:
combining a cannabinoid or an oleoresin, having purity in the range of 0.001% to 100%, an emulsifying or stabilizing agent in sufficient amounts to suspend the cannabinoid or oleoresin within the colloidal mixture to prevent phase separation wherein the cannabinoid or oleoresin remains suspended throughout the final colloidal mixture, and an acidic aqueous solution of pH less than 7.0 sufficient to dissolve the emulsifying or stabilizing agent; and
agitating the combination to create a colloidal mixture wherein the cannabinoid or oleoresin remains suspended throughout the colloidal mixture.

12. The method of claim 11 further comprising the agitation is created by one of mixing, heating, and sonicating.

13. The method of claim 11 further comprising the colloidal mixture is a colloidal suspension or emulsion.

14. The method of claim 11 further comprising the cannabinoid or oleoresin comprises any of alkaloids, glycosides, phenolics, flavonoids, terpenoids, cannabinoids, oleoresins, plant oils, plant resins, plant extracts, plant distillates, plant isolates, and plant raw forms.

15. The method of claim 11 further comprising the emulsifying or stabilizing agent is one of a saccharide, a protein, and a water-soluble pure solid or compound solid mixture.

16. The method of claim 11 further comprising the acidic aqueous solution is one of a fruit juice, a vegetable juice, a plant juice, and purified water with added acid.

17. The method of claim 11 further comprising the acidic aqueous solution comprises any one of citric acid, phosphoric acid, acetic acid, carbonic acid, malic acid, and tartaric acid.

18. The method of claim 11 further comprising the cannabinoid or oleoresin is obtained from one of raw or decarboxylated plant matter, CO₂ extraction, ethanol extraction, butane extraction, butane/propane extraction, hexane extraction, or strained from plant matter soaked in a solvent mixture.

19. The method of claim 11 further comprising, the colloidal mixture is one of dried to a powder, diluted into a drink, mixed with a carrier compound into a topical compound, aerated to form a gel, boiled to a concentrate, and mixed into any food type substance, pharmaceutical, or dietary supplement.
